(19) United States
(12) Patent Application Publication
(10) Pub. No.: US 2026/0020807 A1
(43) Pub. Date: Jan. 22, 2026

(54) CONCURRENT MEASUREMENT OF IMPEDANCE CARDIOGRAPH (ICG) AND ELECTRO CARDIOGRAPH (ECG)

(71) Applicant: Medical Equipment Development, Inc., Houston, TX (US)

(72) Inventor: Hue-Teh Shih, Houston, TX (US)

(21) Appl. No.: 19/275,497

(22) Filed: Jul. 21, 2025

Related U.S. Application Data
(60) Provisional application No. 63/673,965, filed on Jul. 22, 2024.

(51) Int. Cl. A61B 5/304; A61B 5/053; A61B 5/30; A61B 5/308; A61B 5/343

(52) U.S. Cl. CPC A61B 5/304; A61B 5/053; A61B 5/303; A61B 5/308; A61B 5/343

(57) ABSTRACT

Systems and methods for concurrently measuring Impedance Cardiography (ICG) and Electrocardiography (ECG) data are provided. Various embodiments of the present technology provide systems and methods for overcoming limitations of conventional systems by uniquely utilizing a single set of electrodes and cables for both measurements. Embodiments include a system and method that uses a method of connecting a single set of cables and electrodes to a patient's body, injecting an alternating current into the patient's body for ICG measurements while generating no current for ECG measurements, acquiring physiological signals via the single set of cables and electrodes, time-division multiplexing these acquired signals to interleave ICG and ECG data acquisition, and directing the multiplexed signals to distinct first and second processing circuits for ICG and ECG respectively. This approach enables enhanced efficiency, real-time display of virtually synchronized waveforms, and improved verification capabilities for critical physiological events.

### CROSS-REFERENCE TO RELATED APPLICATION(S)

This application claims a benefit of priority under 35 U.S.C. § 119(e) from U.S. Provisional Application No. 63/673,965, filed Jul. 22, 2024, entitled “CONCURRENT MEASUREMENT OF IMPEDANCE CARDIOGRAPH (ICG) AND ELECTRO CARDIOGRAPH (ECG),” which is fully incorporated by reference herein for all purposes.

### TECHNICAL FIELD

This disclosure is directed to systems and methods for obtaining measurements and displaying and/or storing corresponding data for impedance cardiography (ICG) and electro cardiography (ECG), where the same set of cables and electrodes are used for both ICG and ECG.

### BACKGROUND

Electrocardiography (ECG) and Impedance Cardiography (ICG) are established medical techniques used to assess cardiac electrical activity and changes in body fluid status, respectively. ECG records the hearts spontaneous electrical activity, generating a tracing that typically includes a P-wave, QRS complex, and T-wave, whose morphology can vary based on electrode placement. Traditionally, the end of the QRS complex has been used to identify the time when the aortic valve opens, and the end of the T-wave has been used to identify the time when the aortic valve closes. The ECG circuit conventionally includes a rectifier.

ICG involves injecting an alternating current into the patient's body and measuring the received current or change in voltage to calculate impedance based on Ohm's law. This measured impedance is primarily used to represent the fluid status of the body, as water containing electrolytes is the main conductor. Unlike ECG, the ICG processing circuit does not use a rectifier. For ICG measurements, an Analog-to-Digital Converter (ADC) samples the voltage sine wave, and the resistance (R) is calculated from the identified maximum and minimum voltages and currents.

A significant challenge with conventional systems is that conventional ICG and ECG measurements require separate equipment and distinct sets of electrodes and cables. Even when commercially available machines are able display both ICG and ECG, they necessitated two different sets of electrodes and circuits, requiring synchronization by timing if both were to be recorded simultaneously. Furthermore, a critical limitation for ICG has been that the ventricular ejection time, a key parameter for calculating stroke volume, was conventionally derived from the ECG tracing and then applied to the ICG plot, rather than being identified directly from the ICG waveform itself. This reliance on separate hardware and an indirect method for deriving ICG parameters highlights a need for an improved system that enables concurrent and integrated measurement of ICG and ECG using a single set of electrodes and cables, thereby offering enhanced efficiency, direct real-time display, and improved verification capabilities for critical physiological events.

### SUMMARY

The present disclosure describes novel systems and methods that overcome limitations of conventional ICG and ECG measurements by uniquely utilizing a single set of electrodes and cables for concurrent data acquisition through time-division multiplexing, leading to enhanced efficiency and diagnostic capabilities.

Systems and methods are described that measure impedance cardiography (ICG) and electrocardiography (ECG) data by placing a single set of electrodes of a single set of cables on a patient's body, where this single set of cables and electrodes is configured for acquiring both ICG and ECG signals. The method involves injecting an alternating current into the patient's body for ICG measurements, notably with no current generated for ECG measurements. Physiological signals are then acquired from the patient using this single set of cables and electrodes. These acquired signals are then time-division multiplexed to interleave ICG and ECG data acquisition. Finally, the multiplexed signals are directed to either a first processing circuit for ICG measurements (which processes signals originating from the injected current) or a second processing circuit for ECG measurements (which processes spontaneous electrical signals from the heart).

In some embodiments, systems are described that concurrently measure impedance cardiography (ICG) and electrocardiography (ECG) data. Such a system comprises a single set of cables and electrodes configured for placement on a patient's body and for acquiring both ICG and ECG signals. It also includes at least one voltage controlled current source (VCCS) designed to inject an alternating current into the patient's body for ICG measurements, with no current being generated for ECG measurements. A programmable switch is configured within the system to time-division multiplex physiological signals acquired via the single set of cables and electrodes, thereby interleaving ICG and ECG data acquisition. The system further includes a first processing circuit for ICG measurements and a second processing circuit for ECG measurements. The programmable switch is configured to direct the multiplexed signals to either the first processing circuit (which processes signals originating from the injected current) or the second processing circuit (which processes spontaneous electrical signals from the heart).

In some embodiments, methods are described for enabling a system for measuring impedance cardiography (ICG) and electrocardiography (ECG) data. This method involves providing a system that includes a single set of cables and electrodes, where this set is configured for acquiring both ICG and ECG signals. It further comprises configuring at least one current source within the system to inject an alternating current for ICG measurements, ensuring that no current is generated for ECG measurements. The method also includes configuring receiving circuitry within the system to acquire physiological signals using the single set of cables and electrodes. A critical step is programming a controller within the system to perform time-division multiplexing of the acquired physiological signals to interleave ICG and ECG data acquisition. Finally, the method involves providing a first processing circuit for ICG measurements and a second processing circuit for ECG measurements and configuring a switch within the system to direct the multiplexed signals to one of these processing circuits, where the first circuit is configured to process signals originated from the injected current, and the second circuit is configured to process spontaneous electrical signals.

These, and other, aspects of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions and/or rearrangements may be made within the scope of the disclosure without departing from the spirit thereof, and the disclosure includes all such substitutions, modifications, additions and/or rearrangements.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore nonlimiting, embodiments illustrated in the drawings, wherein identical reference numerals designate the same components. Note that the features illustrated in the drawings are not necessarily drawn to scale.

FIG. 1 shows exemplary sine wave voltage and current waveforms.

FIG. 2 shows an exemplary half-wave rectifier circuit diagram.

FIG. 3 shows a high-level conceptual diagram of an example dual-mode ICG and ECG system.

FIG. 4 illustrates interleaved measurements for ICG and ECG using a single set of cables and electrodes, processed by parallel circuits.

FIG. 5 shows a block diagram detailing the components and architecture of a dual-mode ICG and ECG system.

FIG. 6 shows an exemplary circuit diagram for a Voltage Controlled Current Source.

FIG. 7 shows an exemplary circuit diagram for the programmable switch and conceptually illustrates the external electrode connections, test signal output, body surface impedance input, and a calibration circuit.

FIG. 8 shows exemplary circuit diagrams for instrumentation amplifiers used in the ICG and ECG processing circuits.

FIG. 9 shows a circuit diagram for the USB-UART interface.

FIG. 10 shows a flowchart outlining a method for concurrently measuring ICG and ECG data using a single set of electrodes and cables.

FIG. 11 illustrates a data sampling schema and filling of data blanks for a 4:1 ICG to ECG ratio.

FIG. 12 illustrates the data sampling schema and filling of data blanks for a 9:1 ICG to ECG ratio.

### DETAILED DESCRIPTION

The invention and the various features and advantageous details thereof are explained more fully with reference to the non-limiting embodiments that are illustrated in the accompanying drawings and detailed in the following description. Descriptions of well-known starting materials, processing techniques, components and equipment are omitted so as not to unnecessarily obscure the invention in detail. It should be understood, however, that the detailed description and the specific examples, while indicating some embodiments of the invention, are given by way of illustration only and not by way of limitation. Various substitutions, modifications, additions and/or rearrangements within the spirit and/or scope of the underlying inventive concept will become apparent to those skilled in the art from this disclosure.

Generally, the present disclosure describes a system and method for concurrently measuring Impedance Cardiography (ICG) and Electrocardiography (ECG) data, addressing limitations of conventional systems by uniquely utilizing a single set of electrodes and cables for both measurements. In some embodiments, systems are provided which enable a user to take interleaved ICG and ECG measurements using the same hardware. Alternatively, the user can select modes in which ICG measurements alone are taken, or in which ECG measurements alone are taken. Unlike conventional approaches that traditionally require separate equipment and circuits for each modality, the disclosed system employs time-division multiplexing to interleave ICG and ECG data acquisition. A central microprocessor (MCU) with an Analog-to-Digital Converter (ADC) and Universal Asynchronous Receiver/Transmitter (UART) controls the system, allocating acquired samples to their respective data sets via specialized parallel processing circuits—one configured for ICG measurements and another for ECG measurements. This innovative architecture, supported by a programmable switch (relay) and sophisticated software, allows for flexible operation modes, including ICG-only, ECG-only, or a hybrid concurrent mode with configurable sampling ratios (e.g., 4:1 or 9:1 ICG to ECG samples), enabling real-time display of virtually synchronized waveforms and offering enhanced diagnostic capabilities such as improved stroke volume calculation verification and rhythm analysis.

Before describing the systems and methods in detail, the calculation method utilized for ICG will be described, followed by the calculation method utilized for ECG. The relationship of voltage, current and resistance can be written as V(t)=I(t)*R, where V(t) is voltage as a function of time, I(t) is current as a function of time, and R is resistance (impedance). Because I(t) is a controlled parameter, V(t) can be measured, and R then can be calculated.

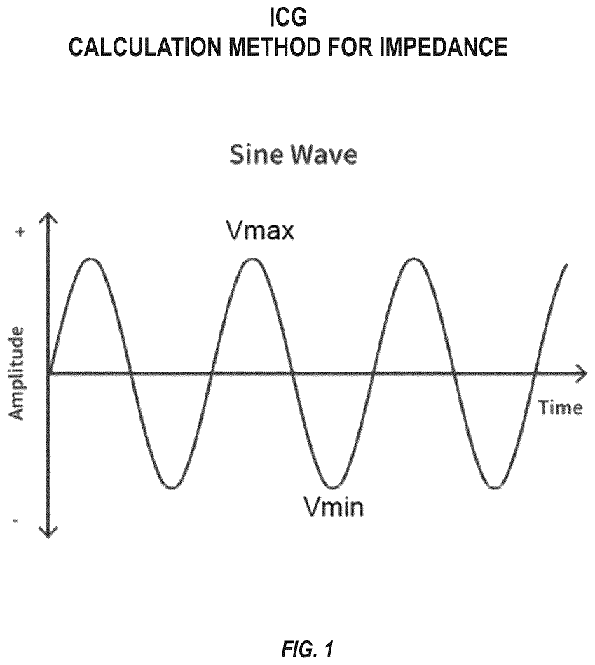

I(t) and V(t) are sine waves (see FIG. 1), and the amplitude of the V(t) sine wave is sampled using an analog-to-digital converter (ADC). The sampling frequency of the ADC should be much higher than the frequency of the sine wave in order to more accurately determine the maximum voltage (Vmax) and minimum voltage (Vmin). If the sampling rate is not high enough, there will be errors introduced on the determined resistance (R) values.

The samples (measurements) are examined to identify the maximum and minimum voltages (Vmax and Vmin), and the difference is divided by the difference between the maximum and minimum currents (Imax and Imin) to determine the resistance (R). In some embodiments, it is difficult for the ADC to process negative voltage, so IA is modified for Vmin>0 and Vmax<3.3 V.

Because the ECG signals resemble an AM waveform, the relative resistance R can be derived by a method similar to AM demodulation, which can capture the envelope of the waveform.

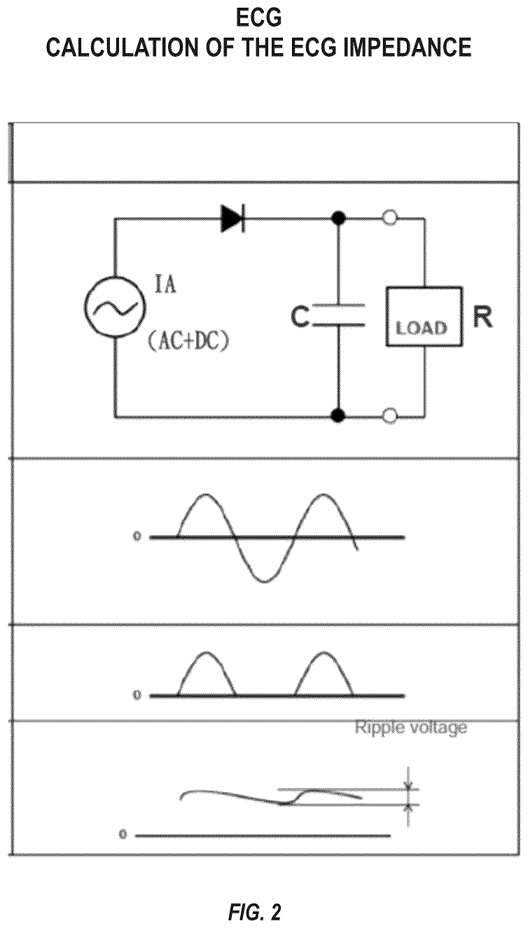

Using rectification of half-waves (see FIG. 2, which shows an exemplary half wave rectifier), the sine wave of AC voltage can be converted to DC voltage (equivalent to the envelope). The R/C values that are carried are calculated and tested in order to minimize the voltage of the ripples, but the time constant cannot be too long, because a time constant that is too long may cause the loss of medium and high frequency components.

Together with an impedance testing board with known resistors, a microprocessor and an ADC continuously sample and obtain the mean voltage of each resistor to establish a table (LUT). When a voltage is actually detected and measured, the value can be interpolated via the table to derive the corresponding resistance value.

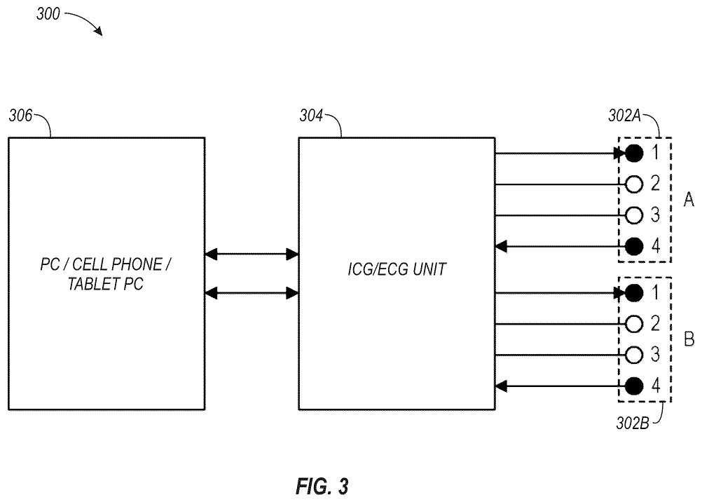

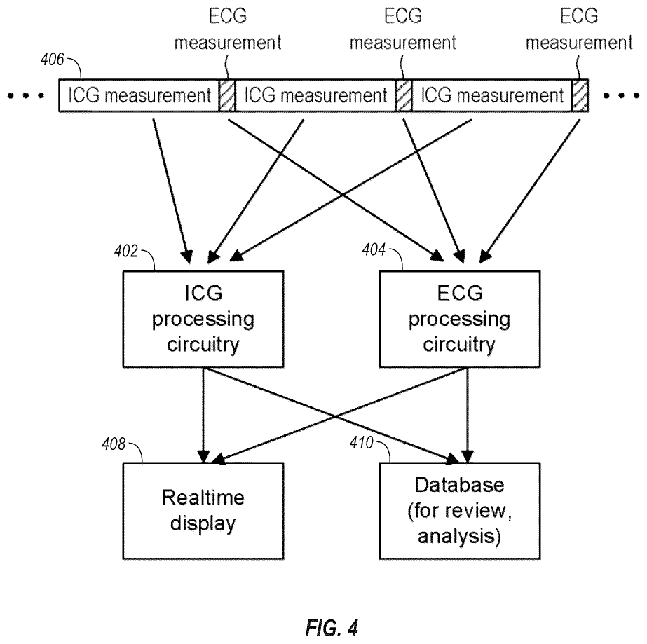

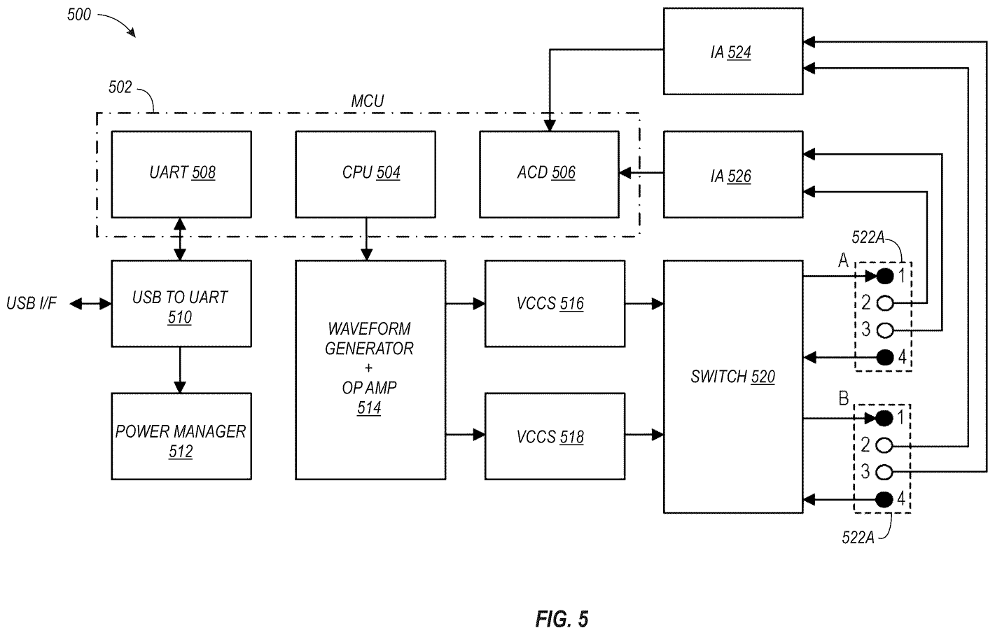

FIG. 3 shows one exemplary high-level conceptual diagram of an example dual-mode ICG and ECG system 300. In the example of FIG. 3, a pair of cables are connected (via cable ports 302A and 302B) to an ICG/ECG unit 304. The ICG/ECG unit 304 is connected through USB and/or UART interfaces to a user device 306 (e.g., a desktop or laptop computer, a cell phone or smart phone, a tablet computer, etc.) A user can provide input (e.g., a desired mode of operation) through a user interface (UI) of the user device 306, which then provides the user input to the ICG/ECG unit 304. The ICG/ECG unit 304 then generates appropriate electrical signals which are applied to particular electrodes of the connected cables (discussed in detail below). The ICG/ECG unit 304 makes measurements on others of the electrodes of the connected cables and processes the measurements to generate ICG and/or ECG data that can be returned to the user device 306 for display to the user and/or stored in a data storage device for further review and/or analysis. While FIG. 3 presents the system at a conceptual level, the detailed operation, such as the interleaved measurement using time-division multiplexing and the specific hardware components (e.g., microprocessor, switch, parallel circuits), are further elaborated with reference to FIGS. 4 and 5, described below.

FIG. 4 is a diagram illustrating interleaved measurements for ICG and ECG using the same equipment (i.e., a single cable having a single set of electrodes). The cable is connected to two parallel sets of processing circuitry 402 and 404. The processing circuitry 402 is dedicated to ICG measurements, which processes signals that originated from the injected current and does not include a rectifier. The processing circuitry 404 is for ECG measurements, designed to process the spontaneous electrical signals from the heart, and includes a rectifier. Two of the electrodes of the cable are used to inject current into a patient's body. Two of the electrodes of the cable are used to receive the returning current.

The system employs a time-division multiplexing approach to interleave ICG and ECG data acquisition. Although the samples are acquired through the same physical connection points, software allocates them to their respective ICG or ECG data sets, causing the waveforms to appear different due to their passage through distinct circuits.

The system takes ICG and ECG measurements at a given sampling rate. In one embodiment, the sampling rate is 2500 Hz (2500 samples per second). In this embodiment, the samples may comprise ICG/ECG samples at a ratio of 9:1 (9 ICG samples to 1 ECG sample), or at a ratio of 4:1 (4 ICG samples to 1 ECG sample). The 9:1 ratio results in an effective ECG sampling rate of 250 Hz, while the 4:1 ratio results in an effective ECG sampling rate of 500 Hz. These effective ECG rates are considered high resolution for ECG.

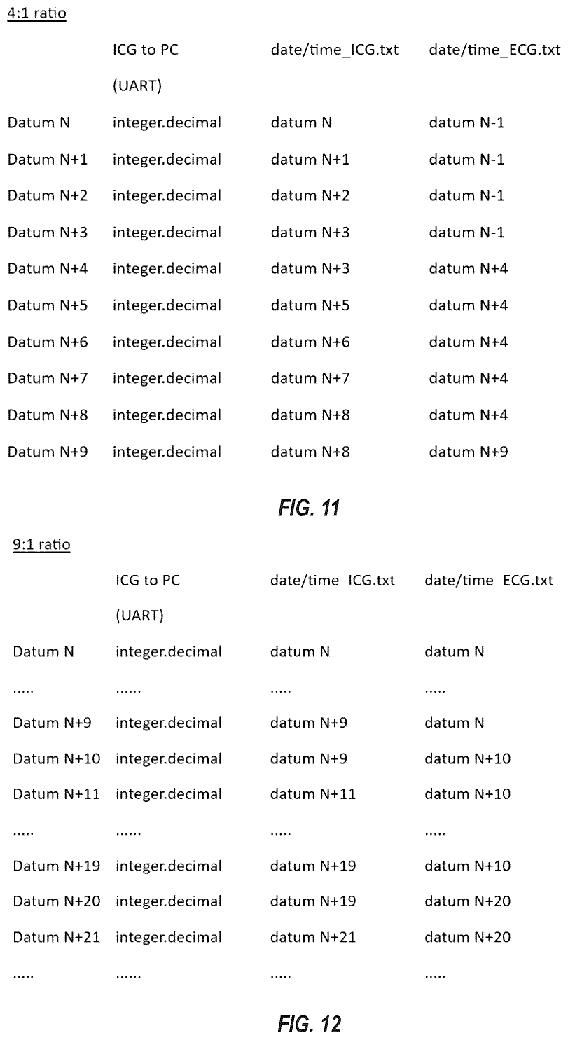

FIGS. 11 and 12 illustrate exemplary data sampling schema and the filling of data blanks when concurrently measuring ICG and ECG data using time-division multiplexing, as described above. This schema matches the time between the two tracings, ensuring a virtually synchronized display. The figures demonstrate configurable sampling ratios, specifically 4 ICG samples to 1 ECG sample (4:1) (FIG. 11) and 9 ICG samples to 1 ECG sample (9:1) (FIG. 12). When data is acquired in these ratios, there will be instances where the ECG column has “blanks” or missing data points to align with the ICG tracing. The schema shown in FIGS. 11-12 details how these blanks can be filled to ensure continuous and synchronized data for display and analysis. This involves using the value from the previously detected ECG point to fill the subsequent blank entries until the next ECG point is acquired.

For example, in a 4:1 ratio (FIG. 11), if four blanks exist in the ECG column following a detected ECG value (e.g., Datum N−1), these blanks (e.g., Datum N, N+1, N+2, N+3 for ECG) are filled with the value from the preceding ECG detection (Datum N−1). When a new ECG point (e.g., Datum N+4) is detected, subsequent blanks are filled with that new value. Similarly, for a 9:1 ratio (FIG. 12), the nine blank entries in the ECG column corresponding to ICG samples would be filled using the value from the last detected ECG data point.

FIG. 4 shows a timeline 406 representing the signal acquisition and the respective portions of time dedicated to ICG measurements and ECG measurements. As is described in more detail below with respect to FIG. 5, signals from the ICG measurement time periods are routed to the ICG processing circuitry 402 and signals from the ECG measurement time periods are routed to the ECG processing circuitry 404. Once the signals are measured, the ones that don't go through the rectifier (relating to ICG measurements) will be the ones that had originated from the current that's been injected to the body. The returning current is received, the voltage is measured, and then the current and voltage are used to calculate the impedance for the ICG. The ICG measurements are made at a certain ratio with respect to the signal that goes through the second (ECG) circuit 404 that has the rectifier. That signal is the signal spontaneously generated by the heart, which is the ECG. Both of the ICG processing circuitry 402 and ECG processing circuitry 404 provide data to the real-time display 408 (e.g., the UI of the user device 306) and the databased 410 (e.g., for review and analysis).

FIG. 5 shows a block diagram of the components and architecture of a dual-mode Impedance Cardiography (ICG) and Electrocardiography (ECG) system 500 in accordance with some embodiments of the invention. FIG. 5 visually outlines how the system 500 achieves concurrent measurement of ICG and ECG using a single set of electrodes and cables, a significant departure from conventional systems.

The system 500 is controlled by a microprocessor (MCU) 502 which includes a central processing unit (CPU) 504 (for example, an ARM Cotex-M3), an ADC 506, and a universal asynchronous receiver/transmitter (UART) 508. The CPU 504 interfaces with external devices, such as desktop computers, laptop computers, cell phones, or tablet computers, via a UART/USB interface 510. This connection allows for command transmission to the system and data transmission from the system for display and analysis. The UART/USB interface 510 also connects the microprocessor 502 to a power manager 512 that supplies power to the entire system 500.

The MCU 502 provides control signals (e.g., via a serial peripheral interface (SPI)) to a waveform generator and an operational amplifier (op amp) 514. The waveform generator 514 generates sine waves at 100 kHz and 5 kHz, which are then adjusted in amplitude by the op amp, and provides each of these generated signals to a corresponding one of two voltage controlled current source (VCCS) units 516 and 518. Both 100 kHz and 5 KHz signals are used for ICG measurements, while no current is generated for ECG measurements. The currents generated by the two VCCS units 516 and 518 are provided to a switch 520 that is configured to switch alternately between the two cable ports 522A and 522B (port A, port B). The utility of the 5 KHz signals is beyond the scope of this disclosure and will not be discussed.

While the system of FIG. 5 depicts two cable ports (522A, 522B) which could enable the use of separate cable sets, this disclosure's primary emphasis and significant advantage lie in its ability to use a single cable set for both ICG and ECG measurements. In this embodiment, the current needed for ICG is applied from one VCCS unit (516 or 518) to the body via a pair of electrodes 1 and 4 (e.g., electrodes corresponding to connection points 1 and 4 of the cable port 522A or 522B), while the signals for ICG and ECG measurements are received via the other pair of electrodes 2 and 3 (e.g., electrodes corresponding to connection points 2 and 3 of the cable port 522A or 522B) and processed alternatingly by 2 parallel circuits (one without a rectifier and the other with a rectifier) respectively.

Each of the cables (via electrodes 2 and 3) is connected to a corresponding instrument amplifier (IA) 524 and 526. Each instrument amplifier is configured to amplify signals for both ICG measurements and ECG measurements. The amplified signals are then provided to the ADC 506 that digitizes samples of the amplified signals and provides the digitized samples to the CPU 504.

As discussed above with respect to FIG. 4, the system 500 employs a time-division multiplexing approach to interleave ICG and ECG data acquisition. A programmable switch (relay), such as switch 520, is controlled by software coding to alternately direct the acquired signals to one of two parallel processing circuits (FIG. 4). While the samples are acquired through the same physical connection points, via a single set of electrodes and cables, software allocates them to their respective ICG or ECG data sets, causing the waveforms to appear different due to their passage through distinct circuits. One processing circuit, for ICG measurements (e.g., circuitry 402), processes signals that originated from the injected current and does not include a rectifier. The other processing circuit, for ECG measurements (e.g., circuitry 404), is designed to process the spontaneous electrical signals from the heart and includes a rectifier. The system takes ICG and ECG measurements at a given sampling rate, such as 2500 samples per second. This allows for configurable ratios of ICG to ECG samples, such as 9:1 (9 ICG samples to 1 ECG sample), which results in an effective ECG sampling rate of 250 Hz, or 4:1 (4 ICG samples to 1 ECG sample), which results in an effective ECG sampling rate of 500 Hz. These effective ECG rates are considered high resolution for ECG. This time-division interleaving creates a “virtually synchronized” display, where the intervals are so small that an operator will not perceive them as staggered. The sampling rate and ratios can be varied, as desired, as one skilled in the art would understand.

The system offers flexible operational modes, allowing a user to select ICG measurements alone, ECG measurements alone, or a hybrid concurrent mode where both are taken simultaneously. This capability represents a significant improvement over conventional systems that typically necessitate separate equipment and distinct sets of electrodes and cables for ICG and ECG measurements.

One benefit of this integrated approach is the enhanced efficiency gained by utilizing a single set of electrodes and cables for both ICG and ECG measurements, addressing a significant challenge in the field. The system provides a direct real-time display of virtually synchronized waveforms. Although the data acquisition for ICG and ECG is staggered in time-division multiplexing, the intervals are so small that an operator will not perceive them as staggered on the screen, appearing virtually synchronized.

Furthermore, the concurrent measurement significantly improves verification capabilities for critical physiological events. Conventionally, the ventricular ejection time (a vital parameter for calculating stroke volume from ICG) was indirectly derived from the ECG tracing by identifying the end of the QRS complex and T-wave, then applying these time points to the ICG plot. With this new system, the ICG waveform can directly identify these points using wavelet software. The concurrent display of ECG then provides a method to verify or double-check the accuracy of the aortic valve opening and closure identified from the ICG waveform. This verification can be performed manually in a review mode on the system's machine.

Another advantage is the ability to assess patient rhythm. While ICG alone can indicate pulse regularity, it cannot provide information about the patient's specific cardiac rhythm. Having a one-channel ECG displayed concurrently allows for this rhythm assessment alongside the ICG data. The system also supports high-resolution ECG by taking ICG and ECG measurements at a sampling rate of 2500 samples per second. Configurable ratios, such as 9 ICG samples to 1 ECG sample, yield an effective ECG sampling rate of 250 Hz, and a 4:1 ratio yields 500 Hz, both considered high resolution for ECG.

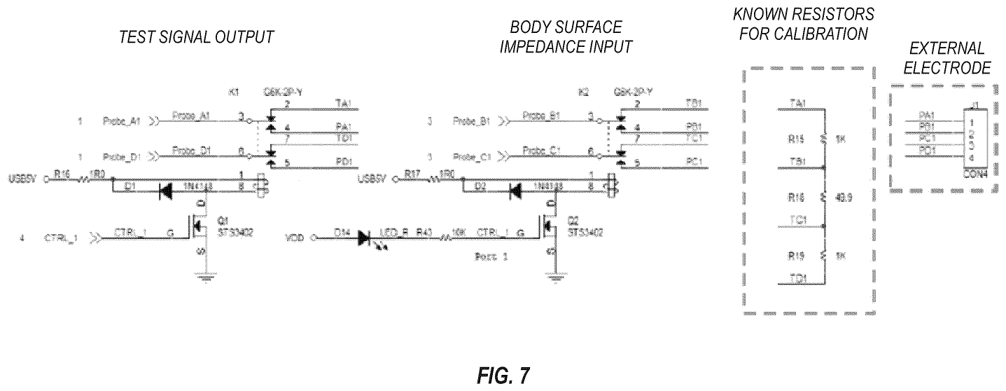

The system also includes a built-in automatic impedance calibration function using known resistors on a calibration circuit (see FIG. 7), which establishes a baseline for improved ICG accuracy. This self-calibration helps correct any obtained values to the known resistor values.

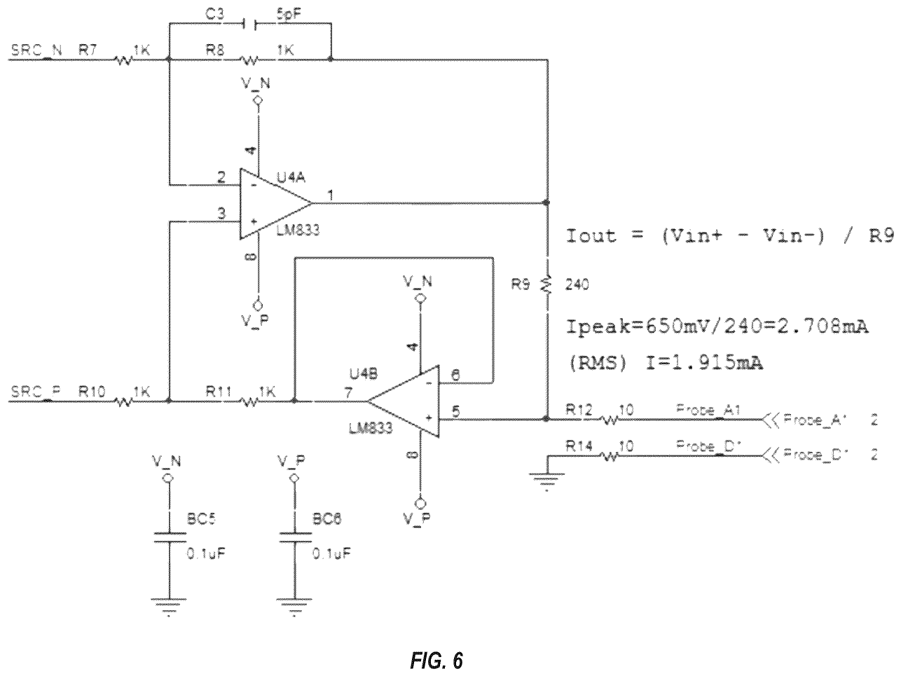

FIGS. 6-9 illustrate exemplary circuit diagrams for the VCCS 516/518, switch 520, instrument amplifiers 524/526 and USB-UART interface 510. While these examples may be used, various alternative circuits could also be used, as one skilled in the art would understand.

FIG. 6 shows an exemplary circuit diagram for a Voltage Controlled Current Source (VCCS). In the dual-mode ICG and ECG system described above, two such VCCS units (516 and 518 in FIG. 5) are utilized. As discussed, these units are responsible for injecting current into the patient's body for ICG measurements via specific electrodes, such as electrodes 1 and 4.

FIG. 7 shows an exemplary circuit diagram for the switch (relay), an integral component (520 in FIG. 5) within the dual-mode ICG and ECG system. This programmable switch is controlled by software coding to facilitate the system's novel time-division multiplexing approach for interleaved ICG and ECG data acquisition. FIG. 7 also conceptually illustrates four sections connected by this switch. FIG. 7 shows the External Electrode that represents the connection to the patient's body via the single set of cables and electrodes (e.g., electrodes 1, 2, 3, and 4) used for both ICG and ECG measurements. FIG. 7 also shows a test signal output, a pathway that allows for the injection of current from the VCCS units into the patient for ICG measurements. FIG. 7 also shows a body surface impedance input, a pathway that is used for receiving the physiological signals from the patient (e.g., via electrodes 2 and 3) for both ICG and ECG measurements. Finally, FIG. 7 shows a calibration circuit containing known resistors, which is used for the system's built-in automatic impedance calibration function to enhance ICG accuracy.

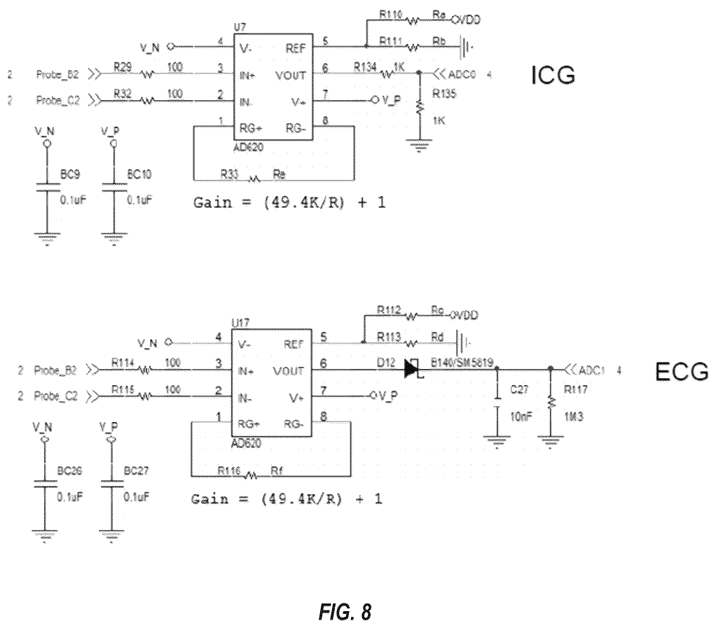

FIG. 8 shows exemplary circuit diagrams for two instrumentation amplifiers (IAs). In the dual-mode ICG and ECG system, each cable (via electrodes 2 and 3) is connected to a corresponding instrument amplifier. These IAs, designated as 524 and 526 in FIG. 5, are configured to amplify signals for both ICG and ECG measurements. FIG. 8 illustrates two separate processing circuits. One processing circuit is dedicated to ICG measurements. This circuit processes signals that originate from the injected current and does not include a rectifier. The other processing circuit is for ECG measurements. It is designed to process the spontaneous electrical signals from the heart and includes a rectifier.

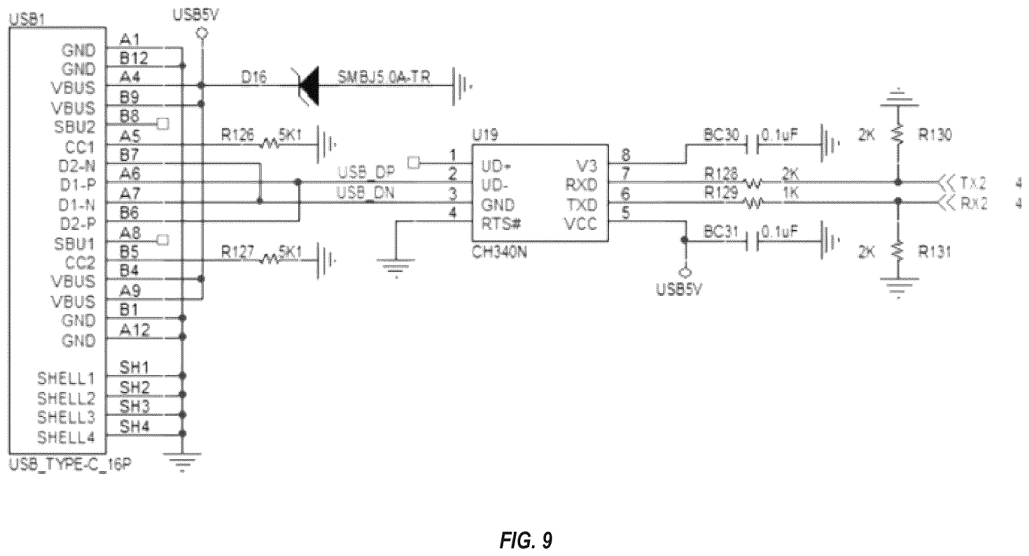

FIG. 9 presents a circuit diagram for the USB-UART interface. This interface (510 in FIG. 5) is a component of the dual-mode ICG and ECG system, enabling the MCU 502 to interface with external devices such as desktop computers, laptop computers, cell phones, or tablet computers. This connection facilitates the transmission of commands to the system and data from the system for display and analysis. In this embodiment, the USB-UART circuit itself is configured in a conventional manner and is based on a USB to UART Bridge Controller CH340 IC, which is a standard component for such functionality.

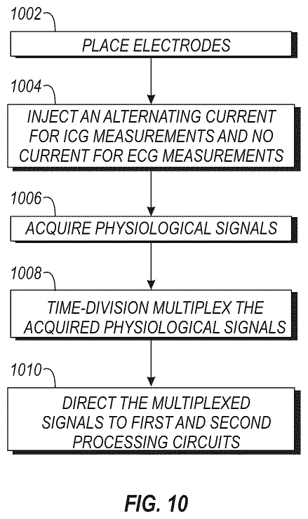

FIG. 10 shows a flowchart that visually outlines a method for concurrently measuring ICG and ECG data. This method uniquely utilizes a single set of electrodes and cables for both measurements, addressing a significant challenge with conventional systems that require separate equipment and distinct sets of electrodes and cables. At step 1002, a single set of cables and electrodes are used, with the electrodes placed on a patient's body. This single set of cables and electrodes is configured for acquiring both ICG and ECG signals. For ICG measurements, typically four electrodes are used: two distal electrodes inject the current, and two proximal electrodes, flanked by the distal ones, serve as receiving electrodes.

At step 1004, an alternating current is injected into the patient's body specifically for ICG measurements, while no current is generated for ECG measurements. As described above, this current is generated by a waveform generator (e.g., producing 100 kHz and 5 kHz sine waves) and then provided to one or more VCCS units, such as VCCS 516 and 518 shown in FIG. 5, which inject the current via particular electrodes (e.g., electrodes 1 and 4). The injected alternating current for ICG typically does not affect ECG measurements due to its nature and the presence of a rectifier in the ECG circuit.

At step 1006, following current injection, physiological signals are acquired from the patient via the single set of cables and electrodes. Signals for both ICG and ECG measurements are received through the same pair of electrodes (e.g., electrodes 2 and 3). These received signals are amplified by instrument amplifiers (described above), which are configured to amplify signals for both ICG and ECG. The amplified signals are then digitized by an ADC. The system is capable of high sampling rates, for example, 2500 samples per second.

At step 1008, the acquired physiological signals undergo time-division multiplexing to interleave ICG and ECG data acquisition. A programmable switch (relay), such as switch 520 (shown in FIGS. 5 and 7), is controlled by software coding to alternately direct the acquired signals. This software-driven process allocates the digitized samples to their respective ICG or ECG data sets, enabling configurable sampling ratios, such as 9 ICG samples to 1 ECG sample (9:1) or 4 ICG samples to 1 ECG sample (4:1). These ratios provide high-resolution effective ECG sampling rates of 250 Hz or 500 Hz. This time-division interleaving results in a “virtually synchronized” display, where the intervals between ICG and ECG acquisition are imperceptible to the operator on screen.

At step 1010, the multiplexed signals are directed to a first processing circuit for ICG measurements or a second processing circuit for ECG measurements. The first processing circuit for ICG measurements (e.g., circuitry 402 in FIG. 4) processes signals that originated from the injected current and does not include a rectifier. For ICG, resistance (impedance) is calculated from the identified maximum and minimum voltages and currents of the sampled sine wave. The second processing circuit for ECG measurements (e.g., circuitry 404 in FIG. 4) processes spontaneous electrical signals from the heart and includes a rectifier. ECG signals resemble an AM waveform, and rectification (e.g., half-wave rectification as shown in FIG. 2) converts the AC voltage to DC voltage to capture the envelope. Despite coming from the same physical connection points, the waveforms appear different due to their passage through these distinct processing circuits. Both processing circuits then provide data for real-time display and storage in a database.

Although the invention has been described with respect to specific embodiments thereof, these embodiments are merely illustrative, and not restrictive of the invention as a whole. Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention.

Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Software implementing embodiments disclosed herein may be implemented in suitable computer-executable instructions that may reside on a computer-readable storage medium. Within this disclosure, the term “computer-readable storage medium” encompasses all types of data storage medium that can be read by a processor. Examples of computer-readable storage media can include, but are not limited to, volatile and non-volatile computer memories and storage devices such as random access memories, read-only memories, hard drives, data cartridges, direct access storage device arrays, magnetic tapes, floppy diskettes, flash memory drives, optical data storage devices, compact-disc read-only memories, hosted or cloud-based storage, and other appropriate computer memories and data storage devices.

Those skilled in the relevant art will appreciate that the invention can be implemented or practiced with other computer system configurations including, without limitation, multi-processor systems, network devices, mini-computers, mainframe computers, data processors, and the like. The invention can be employed in distributed computing environments, where tasks or modules are performed by remote processing devices, which are linked through a communications network such as a LAN, WAN, and/or the Internet. In a distributed computing environment, program modules or subroutines may be located in both local and remote memory storage devices. These program modules or subroutines may, for example, be stored or distributed on computer-readable media, including magnetic and optically readable and removable computer discs, stored as firmware in chips, as well as distributed electronically over the Internet or over other networks (including wireless networks).

Embodiments described herein can be implemented in the form of control logic in software or hardware or a combination of both. The control logic may be stored in an information storage medium, such as a computer-readable medium, as a plurality of instructions adapted to direct an information processing device to perform a set of steps disclosed in the various embodiments. Based on the disclosure and teachings provided herein, a person of ordinary skill in the art will appreciate other ways and/or methods to implement the invention. At least portions of the functionalities or processes described herein can be implemented in suitable computer-executable instructions. The computer-executable instructions may reside on a computer readable medium, hardware circuitry or the like, or any combination thereof.

Any suitable programming language can be used to implement the routines, methods or programs of embodiments of the invention described herein, including C, C++, Java, JavaScript, HTML, or any other programming or scripting code, etc. Different programming techniques can be employed such as procedural or object oriented. Other software/hardware/network architectures may be used. Communications between computers implementing embodiments can be accomplished using any electronic, optical, radio frequency signals, or other suitable methods and tools of communication in compliance with known network protocols.

As one skilled in the art can appreciate, a computer program product implementing an embodiment disclosed herein may comprise a non-transitory computer readable medium storing computer instructions executable by one or more processors in a computing environment. The computer readable medium can be, by way of example only but not by limitation, an electronic, magnetic, optical or other machine readable medium. Examples of non-transitory computer-readable media can include random access memories, read-only memories, hard drives, data cartridges, magnetic tapes, floppy diskettes, flash memory drives, optical data storage devices, compact-disc read-only memories, and other appropriate computer memories and data storage devices.

Particular routines can execute on a single processor or multiple processors. Although the steps, operations, or computations may be presented in a specific order, this order may be changed in different embodiments. In some embodiments, to the extent multiple steps are shown as sequential in this specification, some combination of such steps in alternative embodiments may be performed at the same time. The sequence of operations described herein can be interrupted, suspended, or otherwise controlled by another process, such as an operating system, kernel, etc. Functions, routines, methods, steps and operations described herein can be performed in hardware, software, firmware or any combination thereof.

It will also be appreciated that one or more of the elements depicted in the drawings/figures can be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the meaning of “in” includes “in” and “on” unless the context clearly dictates otherwise.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” “in one embodiment.”

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

Generally then, although the invention has been described with respect to specific embodiments thereof, these embodiments are merely illustrative, and not restrictive of the invention. Rather, the description is intended to describe illustrative embodiments, features and functions in order to provide a person of ordinary skill in the art context to understand the invention without limiting the invention to any particularly described embodiment, feature or function, including any such embodiment feature or function described. While specific embodiments of, and examples for, the invention are described herein for illustrative purposes only, various equivalent modifications are possible within the spirit and scope of the invention, as those skilled in the relevant art will recognize and appreciate.

As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

What is claimed is:

1. A method of measuring impedance cardiography (ICG) and electrocardiography (ECG) data, the method comprising:
placing a single set of electrodes of a single set of cables on a patient's body, the single set of cables and electrodes configured for acquiring both ICG and ECG signals;
injecting an alternating current into the patient's body for ICG measurements, wherein no current is generated for ECG measurements;
acquiring physiological signals from the patient via the single set of cables and electrodes;
time-division multiplexing the acquired physiological signals to interleave ICG and ECG data acquisition; and
directing the multiplexed signals to a first processing circuit for ICG measurements or a second processing circuit for ECG measurements, wherein the first processing circuit for ICG measurements processes signals originated from the injected current, and the second processing circuit for ECG measurements processes spontaneous electrical signals from the heart.

2. The method of claim 1, wherein acquiring physiological signals is performed at a sampling rate of approximately 2500 samples per second.

3. The method of claim 2, wherein time-division multiplexing the acquired physiological signals is performed to achieve a sampling ratio of 9 ICG samples to 1 ECG sample.

4. The method of claim 2, wherein time-division multiplexing the acquired physiological signals is performed to achieve a sampling ratio of 4 ICG samples to 1 ECG sample.

5. The method of claim 2, wherein sampling ratios of ICG samples to ECG samples result in an effective ECG sampling rate of 250 Hz or 500 Hz.

6. The method of claim 1, wherein the time-division multiplexing the acquired physiological signals further comprises controlling a programmable switch with software coding to alternately direct the acquired signals and allocate digitized samples to respective ICG or ECG data sets.

7. The method of claim 1, wherein the first processing circuit for ICG measurements does not include a rectifier, and the second processing circuit for ECG measurements includes a rectifier.

8. The method of claim 1, further comprising displaying ICG and ECG data as virtually synchronized waveforms.

9. A system for concurrently measuring impedance cardiography (ICG) and electrocardiography (ECG) data, the system comprising:
a single set of cables and electrodes configured for placement to a patient's body and for acquiring both ICG and ECG signals;
at least one voltage controlled current source (VCCS) configured to inject an alternating current into the patient's body for ICG measurements, wherein no current is generated for ECG measurements;
a programmable switch configured for time-division multiplexing physiological signals acquired via the single set of cables and electrodes to interleave ICG and ECG data acquisition;
a first processing circuit for ICG measurements and a second processing circuit for ECG measurements, wherein the programmable switch is configured to direct the multiplexed signals to the first processing circuit or the second processing circuit, wherein the first processing circuit is configured to process signals originated from the injected current, and wherein the second processing circuit for ECG measurements is configured to process spontaneous electrical signals from the heart.

10. The system of claim 9, wherein the system is configured for acquiring physiological signals at a sampling rate of approximately 2500 samples per second.

11. The system of claim 10, wherein the programmable switch is configured to perform time-division multiplexing of the acquired physiological signals to achieve a sampling ratio of 9 ICG samples to 1 ECG sample.

12. The system of claim 10, wherein the programmable switch is configured to perform time-division multiplexing of the acquired physiological signals to achieve a sampling ratio of 4 ICG samples to 1 ECG sample.

13. The system of claim 10, wherein the sampling ratios of ICG samples to ECG samples result in an effective ECG sampling rate of 250 Hz or 500 Hz.

14. The system of claim 9, wherein the programmable switch is further configured to be controlled by software coding to alternately direct the acquired signals and allocate digitized samples to respective ICG or ECG data sets.

15. The system of claim 9, wherein the first processing circuit for ICG measurements does not include a rectifier, and the second processing circuit for ECG measurements includes a rectifier.

16. The system of claim 9, further comprising a display configured for displaying ICG and ECG data as virtually synchronized waveforms.

17. A method of enabling a system for measuring impedance cardiography (ICG) and electrocardiography (ECG) data, the method comprising:
providing a system comprising a single set of cables and electrodes, wherein the single set of cables and electrodes is configured for acquiring both ICG and ECG signals;
configuring at least one current source within the system to inject an alternating current for ICG measurements, wherein no current is generated for ECG measurements;
configuring receiving circuitry within the system to acquire physiological signals via the single set of cables and electrodes;
programming a controller within the system to perform time-division multiplexing of the acquired physiological signals to interleave ICG and ECG data acquisition; and
providing a first processing circuit for ICG measurements and a second processing circuit for ECG measurements, and configuring a switch within the system to direct the multiplexed signals to one of the first or second processing circuits, wherein the first processing circuit is configured to process signals originated from the injected current, and the second processing circuit is configured to process spontaneous electrical signals.

18. The method of claim 17, wherein programming the controller within the system to perform time-division multiplexing further comprises programming the controller to control a programmable switch with software coding to alternately direct the acquired signals and allocate digitized samples to respective ICG or ECG data sets.

19. The method of claim 17, wherein the first processing circuit for ICG measurements is configured to not include a rectifier, and the second processing circuit for ECG measurements is configured to include a rectifier.

20. The method of claim 17, further comprising configuring the system to display ICG and ECG data as virtually synchronized waveforms.
